(19) United States
(12) Patent Application Publication
(10) Pub. No.: US 2010/0222293 A1
(43) Pub. Date: Sep. 2, 2010

(54) MODULATION OF SPERM MOTILITY

(76) Inventors: Guglielmo Bonanni, Padova (IT); Marco Rossato, Padova (IT); Francesco Di Virgilio, Padova (IT)

Correspondence Address: FISH & RICHARDSON P.C., P.O. BOX 1022, MINNEAPOLIS, MN, 55440-1022, US

(21) Appl. No.: 12/528,784

(22) PCT Filed: Feb. 27, 2008
(86) PCT No.: PCT/US08/55153

§ 371 Date: May 6, 2010

Related U.S. Application Data
(60) Provisional application No. 60/891,836, filed on Feb. 27, 2007.

(51) Int. Cl. A61K 31/7076; A01N 1/02; C12N 5/02

(52) U.S. Cl. 514/47; 435/2; 435/375

(57) ABSTRACT

Provided are methods of enhancing the fertility of mammalian spermatozoa involving contacting the spermatozoa with adenosine 5′-triphosphate and method of fertilization using spermatozoa treated in this way.

### TECHNICAL FIELD

This invention relates to sperm motility, and more particularly to methods of enhancing sperm motility.

### BACKGROUND

Inadequate sperm motility is a common cause of male mammalian (e.g., human) infertility. Therefore, a simple, non-toxic method of enhancing sperm motility would provide a highly desirable treatment for such in infertility.

### SUMMARY

The inventors have found that in vitro exposure to adenosine 5′-triphosphate (ATP) of spermatozoa from human asthenozoospermic subjects enhanced both progressive velocity and linearity of spermatozoa movement. Thus the invention features methods of enhancing the motility of spermatozoa by exposing them to extracellular ATP and methods of artificial insemination and in vitro fertilization (IVF) using spermatozoa treated with ATP.

More specifically, the invention provides a method of enhancing the motility of a mammalian spermatozoon. The method includes contacting a spermatozoon from a mammalian subject with extracellular adenosine 5′-triphosphate (ATP). Prior to the contacting, a plurality of spermatozoa from the mammalian subject can have been identified as having decreased motility. The mammalian subject can be, for example, a human subject or an equine subject. The spermatozoon can have been obtained from an asthenozoospermic subject. The contacting can include culturing the spermatazoon in a physiological medium with the ATP dissolved therein. Moreover the contacting can be for about 10 minutes to about 120 minutes, e.g., for about 60 minutes. The concentration of extracellular ATP at the initiation of the contacting can be about 100 uM to about 5 mM, e.g., about 2.5 mM. The contacting can be at about 18° C. to about 37° C., e.g., at about 37° C. Prior to the contacting, the spermatozoon can have been substantially separated from seminal plasma. After the contacting, the motility of the spermatozoon can be assessed. Moreover, the contacting can occur in a mammalian female reproductive tract and the mammalian female can be a human or an equine subject.

The invention also features a method of fertilizing a mammalian egg. The method includes contacting a mammalian egg with a mammalian spermatozoon that has undergone the above-described method of enhancing the motility of a mammalian spermatozoon. The contacting of the mammalian egg can occur in vitro and, after the contacting of the mammalian egg and the contacted egg has been fertilized and become an embryo, the mammalian egg can be placed in a mammalian uterus. Alternatively, the contacting of the mammalian egg can occur in vivo, e.g., in a female reproductive tract. The mammalian spermatozoon can be a human spermatozoon and the mammalian egg can be a human egg. In addition, the mammalian spermatozoon can be an equine spermatozoon and the mammalian egg can be an equine egg. The spermatozoon can have been obtained from an asthenozoospermic subject.

As used herein, the term “ATP” refers to adenosine 5′-triphosphate. It includes the acid form as well as salts such as, without limitation, adenosine 5′-triphosphate bis(tris) salt dihydrate (5′-ATP-Bis(TRIS); adenosine 5′-triphosphate dipotassium salt dihydrate (5′-ATP-K₂); adenosine 5′-triphosphate disodium salt (ATP disodium salt); adenosine 5′-triphosphate disodium salt hydrate (ATP disodium hydrate); adenosine 5′-triphosphate magnesium salt (ATP magnesium salt); as well as hydrolysis-resistant ATP analogues such as adenosine 5′-(3-thiotriphosphate) tetralithium salt (ATPγS) and adenosine 5′-(β,γ-imido)triphosphate tetralithium salt (AppNHp, AMP-PNP).

It is understood that, in all the methods of the invention described herein, ATP can be replaced by uridine-5′-triphosphate (UTP) (including chemical forms corresponding to those listed above for ATP).

The terms “spermatozoon” and “spermatozoa” are used interchangeably herein with the term “sperm”.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., enhancing sperm motility, will be apparent from the following description, from the drawings and from the claims.

### DESCRIPTION OF DRAWINGS

FIG. 1 is a diagrammatic representation of the track taken by a motile spermatozoon as observed using an automated cell motility analyzer. The terms VSL, VCL, ALH, VAP, median trajectory, curvilinear trajectory, and rectilinear movement are illustrated in the figure.

FIG. 2 is a series of bar graphs showing the mean straight line velocity (VSL) (FIG. 2A), mean curvilinear velocity (VCL) (FIG. 2B), mean linearity (LIN) FIG. 2C), and lateral head displacement (ALH) (FIG. 2D) of spermatozoa (from 27 asthenozoospermic male human subjects) that had been incubated with or without extracellular ATP for 1 hour. The indicated P values were determined by the Student's t-test from standard deviations.

### DETAILED DESCRIPTION

Based in part on the inventors' findings described above, this document provides methods of enhancing the motility of mammalian spermatozoa and methods of mammalian fertilization using such spermatozoa.

### Methods of Enhancing the Motility of Mammalian Spermatozoa

A method of enhancing the motility of a mammalian spermatozoon is provided. The method involves contacting a spermatozoon from the mammalian subject with a source of extracellular ATP. The spermatozoon is derived from a male subject of the relevant species by methods known in the art including as a component of semen ejaculated by the male subject. Alternatively, sperm can be removed by aspiration from the vas deferens, the epididymis, or the testis of such a male subject. The semen is preferably obtained after a period (e.g., 6 hours, 12 hours, 1 day, 2 days, 3 days, 4 days, 5 days, 7 days, 10 days, 12 days, 14 days or longer) of refraining from ejaculation. Prior to contacting with ATP, the spermatozoon can optionally be washed by standard procedures (e.g., such as the one described in Example 1) so that the contacting of the spermatozoon with ATP occurs in a solution (e.g., culture medium) that is substantially free of seminal fluid. As used herein, “substantially free of seminal fluid” means containing less that 10% (e.g., less than: 8%; 6%; 4%; 2%; 1%; 0.1%; or 0.001%) of seminal fluid-derived components (e.g., proteins, lipids, or nucleic acids) that occur in the seminal fluid from which the spermatozoon is obtained.

The mammalian male subject can be any mammal including a human (e.g., a human patient), a non-human primate (e.g., a monkey, a gorilla, or a chimpanzee), an equine subject (e.g., a horse, a donkey, or a zebra), a pig, a goat, a bovine animal (e.g., a bull), a sheep, a dog, a cat, a rabbit, a guinea pig, a hamster, a gerbil, a rat, or a mouse.

Generally, the mammalian subject will be one whose spermatazoa have lower than normal straight line velocity (progressive velocity) and thus is relatively infertile. As used herein, “normal straight line velocity” is the straight line velocity exhibited by spermatozoa from a fertile male subject of the species (i.e., a subject whose spermatozoa have no evident compromized ability to fertilize an egg of the same species as the spermatozoa). However, it is understood that the methods can also be applied to spermatozoa from apparently entirely normal subjects, e.g., subjects whose spermatozoa have no evident compromized ability to fertilize an appropriate egg but who wish to increase their chances of conceiving.

Prior to the contacting, a plurality of (e.g., 5, 10, 15, 20, 30, 40, 50, 70, 100, 200, 500, 800, 1,000, 2,000, 5,000, 10,000, or even more) spermatozoa from the subject can be optionally tested for motility, i.e., normal, increased, or decreased motility. The spermatazoa tested in this way can be obtained from the same semen sample used to obtain spermatazoa to be contacted with ATP or from a separate sample of semen from the subject. Moreover, after the contacting, the number of spermatazoa can optionally be tested for motility in order to test for the efficacy of the contacting. As used herein, the term “motility” as applied to spermatozoa refers to the straight line velocity or progressive velocity, both designated by the acronym “VSL” (see Example 1). Instead of motility the linearity (i.e., the degree of straightness of the spermatozoon track) can be measured before and after the contacting.

The contacting can be performed in vitro or in vivo. Where it is performed in vitro, the spermatozoon, generally as one of a plurality of spermatozoa, is cultured in a physiological medium (e.g., tissue culture medium) at any of a variety of temperatures (e.g., at about 15° C. to about 39° C., at about 17° C. to about 38° C., at about 18° C. to about 37° C., at about 34° C., at about 35° C., at about 36° C., at about 37° C., or at about 38° C.). As used in the context of these temperatures, the term “about” indicates that the temperature can vary by up to 2° C. from the indicated temperature. The physiological medium can be any culture medium in which mammalian spermatozoa can remain viable and retain their fertilizing potential. Examples of appropriate media include BWW medium or Dulbecco's modified Eagle's Medium (DMEM). Any of a variety of medium supplements can be added to the medium. Such supplements include bacterial and fungicidal antibiotics (e.g., penicillin, streptomycin, gentamycin, and/or amphotericin B) and blood serum from the same species but different individuals as the semen donor (i.e., allogeneic blood serum), blood serum from the semen donor (i.e., autologous blood serum), or blood serum from one or more individuals of a species other than that of the semen donor (i.e., xenogeneic blood serum). Xenogeneic blood serum can be, for example, fetal bovine serum (FBS), equine serum (e.g., horse serum), goat serum, sheep serum, or pig serum. Sera used to supplement tissue culture medium will preferably be allogeneic or autologous blood serum. Thus, where the semen donor is a human, the serum is preferably human serum and where the semen donor is a horse, the serum is preferably horse serum. Where human serum is used, it is preferably from one or more individuals having the AB blood type. Sera to be used as culture medium supplement can also be screened for the presence of anti-spermatozoa antibodies prior to use. Those containing detectable levels of such antibodies would be excluded from use. Additional media supplements include various non-essential or essential amino acids (e.g., glutamine), proteins (e.g., human or bovine serum albumin, insulin, and transferrin), carbohydrates (e.g., sugars such as glucose), nucleic acids, nucleotides, nucleosides, and/or lipids.

The spermatozoon-containing sample of spermatozoa, preferably washed at least once in order to substantially remove seminal fluid, is combined with the culture medium. The ATP can have been added to the medium prior to mixing with the spermatozoa or it can be added after the mixing. The ATP is added so as to give an initial concentration (i.e., the concentration prior to incubation of the ATP/spermatozoon/physiological solution mixture) of about 100 μM to about 10 mM, i.e., about 100 μM to about 5 mM, about 500 μM to about 5 mM, about 1 mM to about 5 mM, about 1 mM to about 2.5 mM, about 1 mM, about 2 mM, about 2.5 mM, or about 3 mM. In the context of these ATP concentrations, the term “about” indicates that the concentration can vary by up to about 10% of the indicated value. The chemical forms of ATP that can be used include all those recited above.

After the spermatozoon, the ATP, and the physiological medium are mixed, the resulting mixture is incubated at one or more of the above listed temperatures for any of a wide range of times, e.g., for about 5 minutes to about 180 minutes, about 10 minutes to about 150 minutes, about 10 minutes to about 120 minutes, about 20 minutes to about 120 minutes, about 30 minutes to 90 minutes, about 30 minutes to about 60 minutes, about 120 minutes, about 90 minutes, about 60 minutes, or about 30 minutes. In the context of these time periods, the term “about” indicates that the time can vary by up to 20% of the indicated value. After the incubation, the spermatozoon-containing population of spermatozoa can be used for any of a variety of fertilization procedures (see below), optionally after washing with a physiological solution, e.g., normal saline, phosphate-buffered saline (PBS), or tissue culture medium.

Contacting of a spermatozoon with ATP in vivo can be achieved by delivering a composition containing ATP and a spermatozoon-containing sample of spermatozoa into a female reproductive tract. Thus, for example, prior to, or soon after, sexual intercourse between a male and female subject of a species of interest, ATP-containing composition can be inserted into the vagina of the female subject. Alternatively, semen, or spermatozoa washed substantially free of seminal fluid, can be artificially infused or injected into the female subject's vagina. Generally, the composition will be inserted no more than about 60 minutes, about 45 minutes, 30 minutes, 20 minutes, 10 minutes, 5 minutes, 2 minutes, or 1 minute before or after the spermatozoon is delivered to the female genital tract. In the context of these time periods, the term “about” indicates that the time can vary by up to 10% of the indicated value. In these methods a spermatozoon delivered to the female vagina comes into contact with the ATP. Naturally, the species of female can be any of the species listed above as donors of semen as a source of the spermatozoon. The male that is the source of spermatozoon and the female into whose genital tract the spermatozoon is delivered are generally, but not necessarily, of the same species.

The ATP compositions that are delivered to the female genital tract (either by for example, application, infusion, or injection) can be in the form, e.g., of a powder, a granule, a tablet, a capsule, a troche, a liquid (e.g., a lotion), a gel, an ointment, a cream, or an emulsion. In a liquid, a gel, an ointment, a cream, or an emulsion, the ATP can be dissolved or in suspension and will generally be present in the composition at the same concentrations listed above for in vitro contacting of the spermatozoon with ATP. The compositions typically include the ATP and a pharmaceutically acceptable carrier. As used herein, “pharmaceutically acceptable carriers” include solvents, dispersion media, coatings, antibacterial and antifungal agents, isotonic and absorption delaying agents, and the like, compatible with pharmaceutical administration. Supplementary active compounds can also be incorporated into the compositions.

The composition is preferably sterile. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. In the case of powders, the methods of preparation can include vacuum drying or freeze-drying which yields a powder of the active ingredient plus any additional desired ingredient from a solution thereof.

The powders, granules, tablets, capsules, and troches contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it.

Aqueous solutions can be prepared by dissolving the ATP in water and adding suitable colorants, stabilizers, and thickening agents as desired. Aqueous suspensions can be made by dispersing the finely divided active component in water with viscous material, such as natural or synthetic gums, resins, methylcellulose, sodium carboxymethylcellulose, and other well-known suspending agents.

The compositions can also be prepared in the form of suppositories (e.g., with conventional suppository bases such as cocoa butter and other glycerides) or retention enemas for vaginal delivery.

In one embodiment, the ATP is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It can be advantageous to formulate compositions in dosage unit form for ease of administration and uniformity of dosage. Dosage unit form as used herein refers to physically discrete units suited as unitary dosages for the subject to be treated; each unit containing a predetermined quantity of ATP calculated to produce the desired motility-enhancing effect in association with the required pharmaceutical carrier. Dosage units can also be accompanied by instructions for use.

This document also provides an article of manufacture, which includes a container and/or delivery vehicle (e.g., a bottle, vial, capsule, or a tablet) containing ATP and packaging material and/or a package insert containing instructions on how to perform any of the above-described methods of enhancing the motility of spermatozoa.

### Methods of Fertilization

As indicated above, a spermatozoon in a population of spermatozoa that have been contacted in vitro with ATP can be used in any of a variety of IVF and artificial insemination procedures known in the art. Examples of such procedures are described in detail in Hall et al. (1997) Baillieres Clin. Obstet. Gynaecol. 11:711-24; Van Steirteghem (1994) Curr. Opin. Obstet. Gynecol. 6:173-177; and Van Voorhis (2007) N. Engl. J. Med. 356:379-386, the disclosures of which are incorporated herein by reference in their entirety.

Prior to use in these procedures, the spermatozoa can optionally (but not necessarily) be washed so as to be free or substantially free of ATP-containing physiological medium. As used herein, “substantially free of ATP-containing physiological medium” means contains less that 10% (e.g., less than: 8%; 6%; 4%; 2%; 1%; 0.1%; or 0.001%) of the ATP-containing physiological medium used for contacting the spermatozoon with ATP.

In essence, IVF procedures involve incubating an egg from a female subject of any of the above-listed species with a spermatozoon from a male subject of any of the same species and which has undergone the above described motility-enhancing method. After the incubation, one or more embryo(s) developed after sperm penetration of one or more eggs are inserted into the uterus of a female subject, generally, but not necessarily, the subject from which the egg was originally obtained. Again, the male donor of the spermatozoon/oa) and the female donor of the egg(s) can be of any of the species listed above. Usually, but not always, the male and female are of the same species. Generally, 16-24 hours after penetration of an egg by a sperm, there is fusion of the sperm and egg nuclei (i.e., the formation of a zygote). About 48 hours after sperm penetration of the egg, there is formation of an embryo, i.e., a structure generally containing two cells. The embryo can be transferred to uterus of the female subject at this stage or at a later stage of embryo maturation (up to five days after sperm penetration of the egg). Prior to the transfer, fertilization of the egg and subsequent embryo formation can be tested for by methods familiar to those in the art.

Artificial insemination procedures involve, for example, delivering a spermatozoon that has undergone the above-described motility enhancing procedure to the reproductive tract (e.g., the vagina and/or uterus) of a female subject. In this case it is hoped that the spermatozoon will come in contact with an egg produced by the female subject and fertilize it. Again, the male donor of the spermatozoon and the female subject can be of any of the species listed above. Generally, but not always, the male and female are of the same species.

The following Examples serve to illustrate, not limit, the invention.

### EXAMPLES

### Example 1

### Materials and Methods

### Sperm Motility

Sperm motility was graded on a scale of a to d, according to the World Health Organisation (WHO) Laboratory Manual [World Health Organization (1999) WHO laboratory manual for the examination of human semen and sperm-cervical mucus interaction, p. 128. Cambridge University Press, Cambridge, the disclosure of which is incorporated herein by reference in its entirety] criteria, as follows:

grade a (fast progressive) sperm that swim forward fast in a straight line;

grade b (slow progressive) sperm swim forward, but either in a curved or crooked line, or slowly (slow linear or non-linear motility);

grade c (nonprogressive) sperm move their tails, but do not move forward (local motility only); and

grade d (immotile) sperm do not move at all.

“Fast” and “slow” are qualitative characteristics of sperm movement defined by the operator examining the samples based on his or her experience at examining spermatozoa.

Low percentages of grade a and b motility and high percentages of grade c and d are considered as indicators for poor sperm fertilizing ability.

### Patient Selection

Sperm from twenty seven asthenozoospermic, otherwise healthy, men referred to the Andrological Center (University of Padova, Department of Medical and Surgical Sciences, Section of Endocrinology) because of infertility were analyzed. As used herein the term “asthenozoospermic” refers to male subjects whose percentage of sperm having motility grade a plus percentage of sperm having grade b are lower than 50%. Cultures of aliquots of the semen samples from all subjects showed no bacterial contamination of the samples and no anti-sperm antibodies were detected in the sera of any of the subjects.

Sperm characteristics were as follows:

Sperm motility: percent grade a+percent grade b (as evaluated by light microscopy) <50%.

Sperm concentration in semen: ≧20×10⁶/ml

Sperm with normal morphology: ≧30%

Sperm viability: ≧50%

### Experimental Protocol

Semen samples were collected after 3 days of sexual abstinence in sterile containers. After liquefaction at room temperature for 30 minutes, standard seminal parameters were examined according to the WHO laboratory manual [supra]. For sperm movement analysis, a semen aliquot (10 μl) was placed in a Makler chamber, ten different fields per chamber were randomly examined, and at least 100 sperm for each field of the chamber were scored. Percentages of motile sperm and movement characteristics were analysed using an automated analyzer at 37° C. (CellTrack VP110, Motion Analysis Corporation, Palo Alto, Calif., USA). Sperm velocity and kinetic parameters were evaluated for only motile sperm and were expressed as mean values. Standard deviations were calculated and P values of significance were determined by the Student's t-test.

The following sperm parameters were determined:

curvilinear velocity (VCL) (also referred to as track speed and is the velocity (in μm/sec) of a sperm along individual tracks;

straight progressive velocity (VSL) (the average velocity (in μm/sec) measured in a straight line from the beginning to the end of track);

amplitude of lateral head displacement (ALH) (mean width (in μm) of sperm head oscillation); and

linearity coefficient (LIN=VSL/VCL×100) (the degree (in %) of straightness of the sperm track).

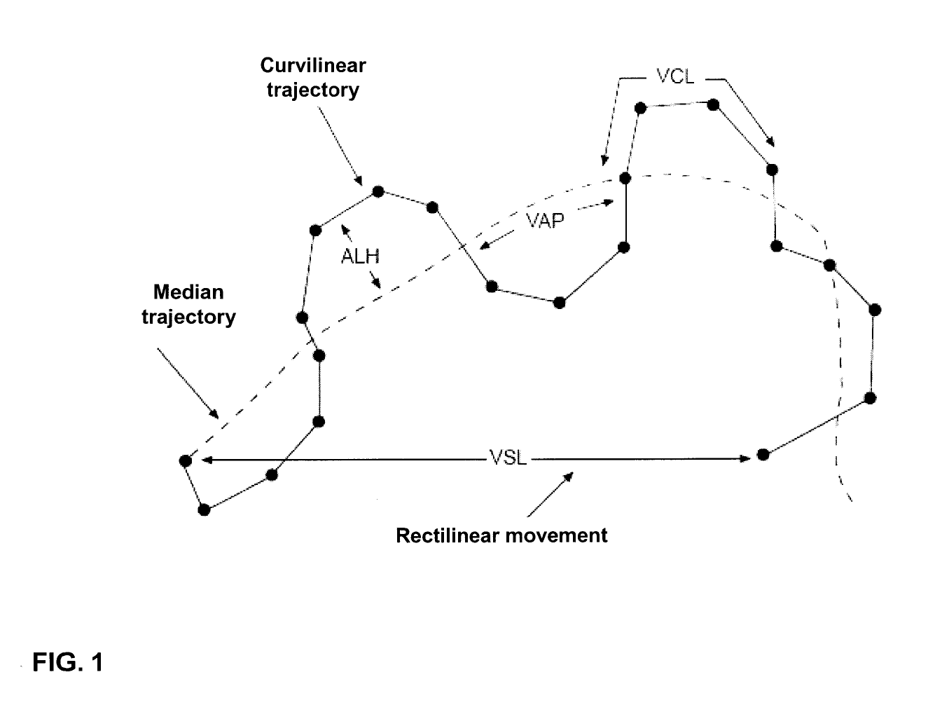

These parameters are illustrated diagrammatically in FIG. 1. In FIG. 1, VAP refers to the average velocity of the smoothed sperm path (μm/sec).

To analyze the effects of extracellular ATP on motility parameters of sperm from the asthenozoospermic subjects, sperm samples from the subjects were washed once by the addition of BWW medium (Biggers-Whitten-Whittingham medium; 95 mM NaCl, 4.8 mM KCl, 1.7 mM CaCl₂, 1.2 mM KH₂PO₄, 1.2 MgSO₄, 25 NaHCO₃, 5.6 mM fructose, 0.25 mM sodium pyruvate, 3.7 ml/l 60% sodium lactate syrup, 10⁴ IU/ml penicillin, and 10 mg/ml streptomycin) and centrifugation at 800×g for 10 minutes. After centrifugation and discarding of the supernatant, the sperm pellets were suspended in BWW medium (at a concentration of 10×10⁶ sperm/ml), divided into two equivalent aliquots, and allowed to recover for 15 minutes. One aliquot was then incubated at 37° C. with ATP (2.5 mM final concentration from a 100 mM stock solution made by diluting ATP with physiological solution (0.9% NaCl in distilled water) and the second aliquot was incubated at 37° C. with same amount of physiological solution only (as a negative control). After 60 minutes incubation, sperm motility parameters were determined as described above.

### Example 2

### Treatment of Sperm from Asthenozoospermic Subjects with Extracellular ATP

The effects of extracellular ATP on sperm motility parameters of sperm from subjects with asthenozoospermia after washing from seminal fluid and resuspension in BWW medium (10×10⁶ sperm/ml) were examined.

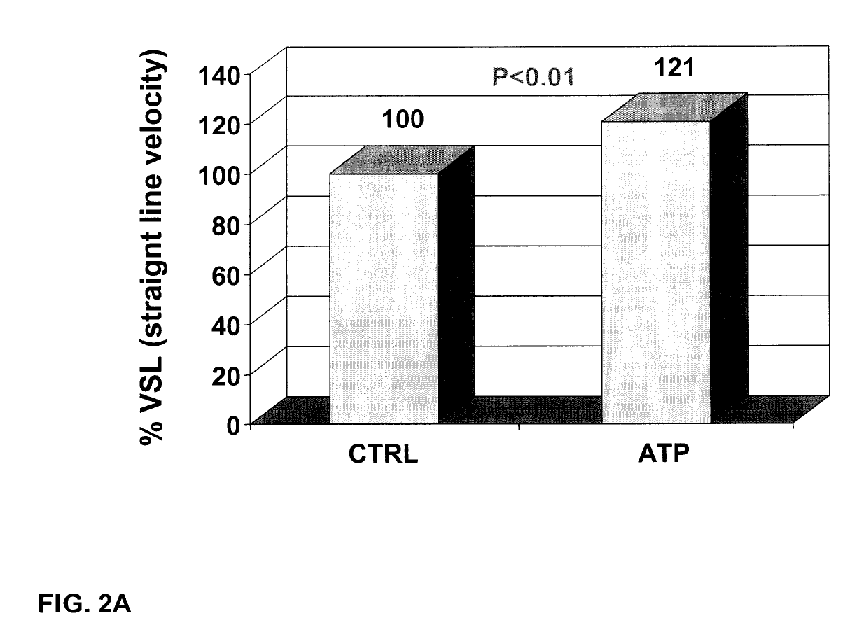

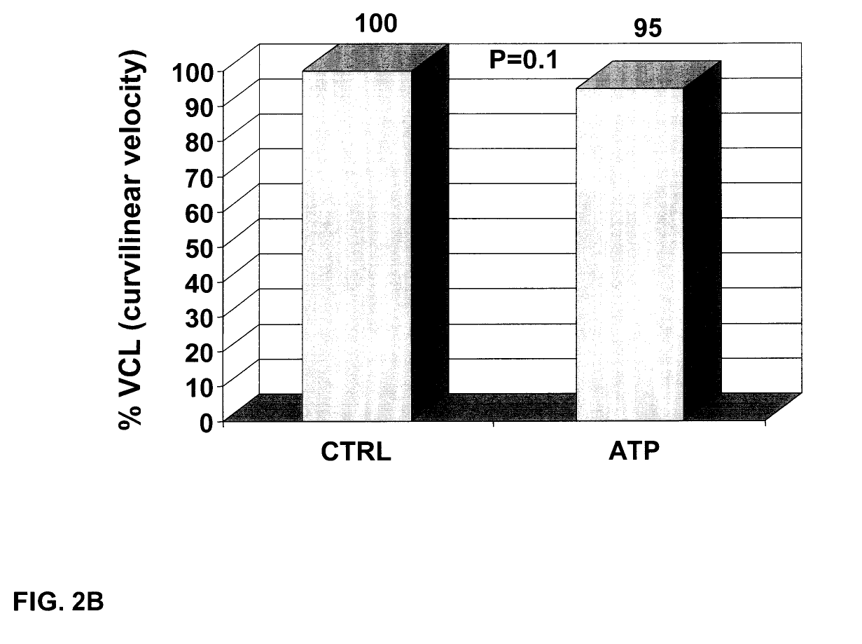

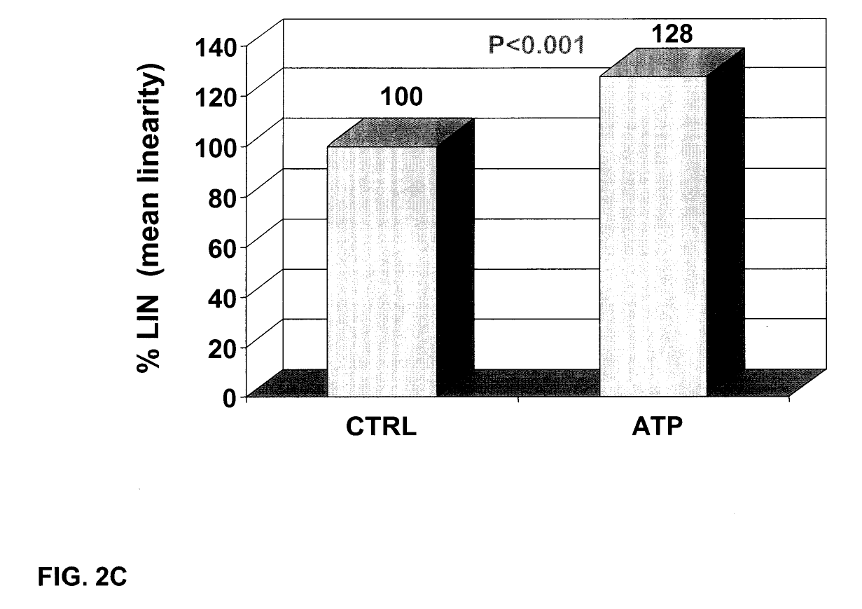

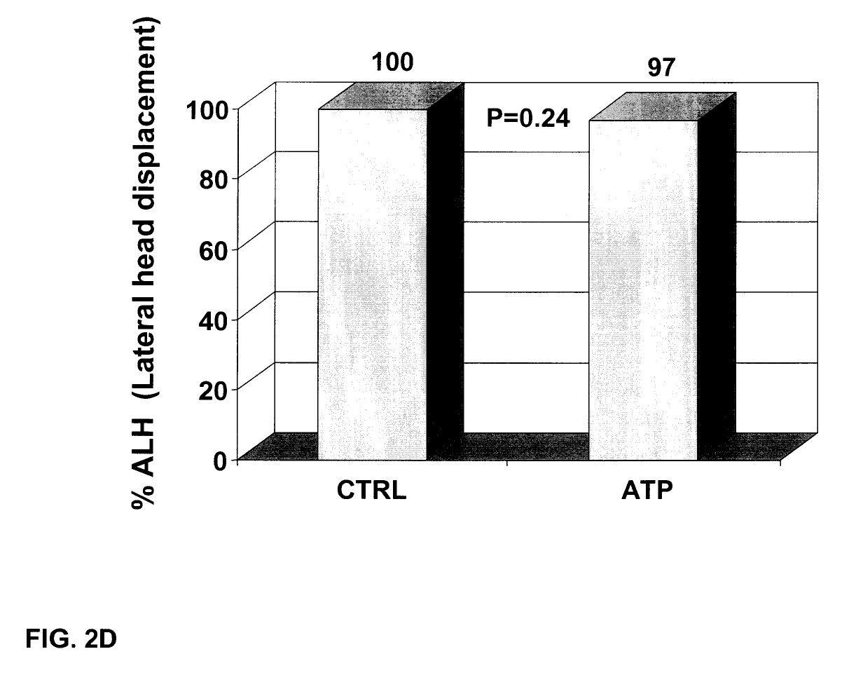

Incubation of sperm of asthenozoospermic subjects in the presence of 2.5 mM ATP for 60 minutes modified sperm motility parameters including progressive velocity (control 23.81±6.04 μM/sec, ATP 28.85±7.27 μm/sec; p=0.0024) and linearity (control 29.59±6.25, ATP 37.7±7.9; p=0.0004) (FIG. 2). No significant effects of ATP treatment were observed on curvilinear velocity and lateral sperm head displacement after ATP treatment (2.5 mM).

Thus, it appears that ATP treatment of sperm from asthenozoospermic subjects improves sperm motility characteristics. These findings provide mechanistic support for how ATP improves human IVF success and indicate that exposure of sperm incubation to extracellular ATP can be a useful and non toxic treatment for infertility.

A number of embodiments of the invention have been described. Nevertheless, it will be understood that various modifications may be made without departing from the spirit and scope of the invention. Accordingly, other embodiments are within the scope of the following claims.

1. A method of enhancing the motility of a mammalian spermatozoon, the method comprising contacting a spermatozoon from a mammalian subject with extracellular adenosine 5′-triphosphate (ATP).

2. The method of claim 1, wherein, prior to the contacting, a plurality of spermatozoa from the mammalian subject are identified as having decreased motility.

3. The method of claim 1, wherein the mammalian subject is a human subject.

4. The method of claim 1, wherein the mammalian subject is an equine subject.

5. The method of claim 1, wherein the contacting comprises culturing the spermatazoon in a physiological medium with the ATP dissolved therein.

6. The method of claim 1, wherein the contacting is for about 10 minutes to about 120 minutes.

7. The method of claim 1, wherein the contacting is for about 60 minutes.

8. The method of claim 1, wherein the concentration of extracellular ATP at the initiation of the contacting is about 100 uM to about 5 mM.

9. The method of claim 1, wherein the concentration of extracellular ATP at the initiation of the contacting is about 2.5 mM.

10. The method of claim 1, wherein the contacting is at about 18° C. to about 37° C.

11. The method of claim 1, wherein the contacting is at about 37° C.

12. The method of claim 1, wherein prior to the contacting, the spermatozoon is substantially separated from seminal plasma.

13. The method of claim 1, wherein, after the contacting, the motility of the spermatozoon is assessed.

14. The method of claim 1, wherein the contacting occurs in a mammalian female reproductive tract.

15. The method of claim 14, wherein the mammalian female is a human.

16. The method of claim 14, wherein the mammalian female is an equine subject.

17. A method of fertilizing a mammalian egg, the method comprising contacting a mammalian egg with a mammalian spermatozoon that has undergone the method of claim 1.

18. The method of claim 17, wherein the contacting of the mammalian egg occurs in vitro.

19. The method of claim 17, wherein, after the contacting of the mammalian egg and the contacted egg has been fertilized and become an embryo, the mammalian egg is placed in a mammalian uterus.

20. The method of claim 17, wherein the contacting of the mammalian egg occurs in vivo.

21. The method of claim 20, wherein the contacting of the mammalian egg occurs in a female reproductive tract.

22. The method of claim 17, wherein the mammalian spermatozoon is a human spermatozoon and the mammalian egg is a human egg.

23. The method of claim 17, wherein the mammalian spermatozoon is an equine spermatozoon and the mammalian egg is an equine egg.

24. The method of claim 1, wherein the spermatozoon is obtained from an asthenozoospermic subject.

25. The method of claim 17, wherein the spermatozoon is obtained from an asthenozoospermic subject.
